(19) United States
(12) Patent Application Publication
(10) Pub. No.: US 2016/0303178 A1
(43) Pub. Date: Oct. 20, 2016

(54) PHARMACEUTICAL COMPOSITION, METHOD FOR PREPARING THE SAME AND USE THEREOF

(71) Applicant: HUMANWELL HEALTHCARE (GROUP) CO., LTD., Wuhan, Hubei (CN)

(72) Inventors: XUEHAI WANG, Wuhan, Hubei (CN); JIE LI, Wuhan, Hubei (CN); LI'E LI, Wuhan, Hubei (CN); YONG XU, Wuhan, Hubei (CN); RONGHUA TU, Wuhan, Hubei (CN); GANG CHEN, Wuhan, Hubei (CN); RUBIN CAO, Wuhan, Hubei (CN); YUN FENG, Wuhan, Hubei (CN); ZHONGWEN YANG, Wuhan, Hubei (CN); ZHAOZE FAN, Wuhan, Hubei (CN); YANPING YU, Wuhan, Hubei (CN); QIANG XIAO, Wuhan, Hubei (CN); LU HUANG, Wuhan, Hubei (CN); CHENGBING YANG, Wuhan, Hubei (CN); TIANCI HUANG, Wuhan, Hubei (CN); HUA TIAN, Wuhan, Hubei (CN); JING YANG, Wuhan, Hubei (CN)

(21) Appl. No.: 15/101,917

(22) PCT Filed: Jul. 15, 2014
(86) PCT No.: PCT/CN2014/082233

§ 371 Date: Jun. 5, 2016

(30) Foreign Application Priority Data
Dec. 5, 2013 (CN) 201310643172.6

(51) Int. Cl. A61K 36/48; A61K 31/353; A61K 9/16; A61K 9/48; A61K 9/28

(52) U.S. Cl. CPC A61K 36/48; A61K 9/4833; A61K 9/2893; A61K 9/1682; A61K 31/353; A61K 2236/33

(57) ABSTRACT

Disclosed are a pharmaceutical composition, a method for preparing the same and use thereof. The pharmaceutical composition consists of total flavonoids of Desmodium Styracifolium as an active ingredient and a pharmaceutically acceptable excipient. The pharmaceutical composition can be formulated in a form of a medicament formulation suitable to administration in clinic, and can be used in preparation of clinic treatment medicaments for treating dampness-heat and urinary stone (stagnation of dampness-heat).

### FIELD

The present disclosure relates to the field of Chinese medicine, and particularly to a pharmaceutical composition, a method for preparing the same and use thereof. More particularly, the present disclosure provides a pharmaceutical composition, use of the pharmaceutical composition in preparation a medicament and a method for preparing the pharmaceutical composition.

### BACKGROUND

Desmodium Styracifolium is a dried overground part of leguminous plants, Desmodium styracifolium (Osb.) Merr., as a traditional Chinese medicine recorded in Part I of Chinese Pharmacopoeia (2010 edition) having efficacy in disinhibiting dampness-abating jaundice and disinhibiting urine and freeing strangury. A prescription preparation of stranguria-treating and calculus-removing tablet containing Desmodium Styracifolium as its essential ingredient, also recorded in Chinese Pharmacopoeia, can be used to treat bladder dampness-heat, stone strangury with roughness and pain in the urethra, lithangiuria and urinary infection belonging to dampness and heat in liver, gallbladder and urinary bladder. However, the raw material of the stranguria-treating and calculus-removing tablet is a crude extract of the Desmodium Styracifolium which is prepared by a traditional water-extraction and alcohol-precipitation extraction method, and this tablet also has a plurality of drawbacks, such as unclear effective components in Chinese herb, overdose in clinic (6 times a day, three pills one time, sugar-coated tablets or film-coated tablets, each pill containing 0.12 g dry extract) and inadequate standard in quality control. The stone discharging agent, such as potassium citrate, thiazide diuretic, magnesium agent, and acetyl cysteine, which is often used in clinic to treating the lithangiuria, with an non-ideal efficacy and significant toxicity and side effect. Chinese patent medicine, such as “Mi Shi Tong”, lithagogue infusion, and stranguria-treating and calculus-removing tablets, is commonly used medicaments with exact effect. However, similar with the stranguria-treating and calculus-removing tablets, all these traditional Chinese medicines still exist such problems, such as original pharmaceutical process, difficulties in quality control, inaccurate quantitative detection method, and overdose, that there is a relative great distance as compared with international standards and does not meet the requirements of modern clinical medicine.

Currently, the medicament for treating urinary stone still needs to be further improved.

### SUMMARY

Embodiments of the present disclosure seek to solve at least one of the problems existing in the related art to at least some extent or to provide at least one of commercially available choices. Therefore, in view of a situation that the medicament for treating urinary stone is in short currently, the present disclosure provides a pharmaceutical composition, a method for preparing the same and use thereof.

In a first aspect, a pharmaceutical composition is provided. The pharmaceutical composition consists of total flavonoids extract of Desmodium Styracifolium as an active ingredient and a pharmaceutically acceptable excipient, in which the total flavonoids of Desmodium Styracifolium is provided in a form of alcohol extract of Desmodium Styracifolium, and is of a weight percentage ranging from 2.5% to 95%, based on a total weight of the pharmaceutical composition.

In the pharmaceutical composition of the present disclosure, the pharmaceutically acceptable excipient is at least one selected from corn starch, dextrin, lactose, pregelatinized starch, saccharose, microcrystalline cellulose, mannitol, sorbitol, xylitol, calcium hydrophosphate, calcium carbonate, starch paste, hydroxypropyl methyl cellulose, povidone K₃₀, povidone K₂₅, polyethylene glycol 2000, polyethylene glycol 4000, polyethylene glycol 6000, citric acid, succinic acid, dextran, galactose, saccharose, glucose, modified starch, microcrystalline cellulose, poloxamer 188, D-mannitol, methylcellulose, sodium carboxymethyl starch, low-substituted hydroxypropyl cellulose, cross-linked povidone, sodium carboxymethyl starch, croscarmellose sodium, calcium carboxymethyl cellulose, coconut oil amine polyglycol ether, glycerol polyoxyethylene ether, Tween 20, Tween 40, Tween 60, Tween 80, Myrj 40, Brij 30, methoxy polyethylene glycol, sodium dodecyl sulfate, magnesium stearate, talc, aerosil, magnesium dodecyl sulfate, sodium benzoate, and sodium stearyl fumarate.

The urinary stone disease can be effectively treated by the present pharmaceutical composition.

In an embodiment of the present disclosure, the total flavonoids extract of Desmodium Styracifolium is prepared by the following steps: extracting a raw material of Desmodium Styracifolium with ethanol, so as to obtain an extracting solution of Desmodium Styracifolium, and purifying the extracting solution of Desmodium Styracifolium, so as to obtain the total flavonoids extract of Desmodium Styracifolium. In an embodiment of the present disclosure, resulting total flavonoids extract of Desmodium Styracifolium can be dried to obtain a solid product as required.

In an embodiment of the present disclosure, extracting the raw material of Desmodium Styracifolium with ethanol further includes: heating and refluxing the raw material of Desmodium Styracifolium for extraction, 1 to 3 times with 1 to 3 hours for each time, with ethanol having a concentration ranging from 50% to 95% and a weight ranging from 8 to 14 times as heavy as the raw material of Desmodium styracifolium, to obtain alcohol extracting solutions of Desmodium styracifolium followed by mixing, so as to obtain the extracting solution of Desmodium styracifolium. In a specific embodiment of the present disclosure, purifying the extracting solution of Desmodium Styracifolium further includes: concentrating the extracting solution of Desmodium Styracifolium, so as to remove ethanol; and subjecting the extracting solution of Desmodium Styracifolium after concentrated to adsorption onto a macroporous resin column, so as to obtain purified total flavonoids extract of Desmodium Styracifolium.

In specific, in an embodiment of the present disclosure, a method for preparing the total flavonoids extract of Desmodium Styracifolium includes the following steps:

heating and refluxing a raw material of Desmodium Styracifolium for extraction, 1 to 3 times with 1 to 3 hours for each time, at a temperature of 50° C. to 60° C. with ethanol having a concentration of 50% to 95% and a weight ranging from 8 to 14 times as heavy as the raw material, so as to obtain alcohol extracting solutions of Desmodium Styracifolium followed by mixing;

concentrating the alcohol extracting solution to be of a volume 2 to 8 times the weight of the raw material followed by still standing and filtering to obtain a filtrate;

subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and

concentrating the eluted solution to recycle ethanol and to obtain a concentrated solution with a relative density ranging from 1.10 to 1.30 followed by drying and then smashing so as to obtain fine powder of the total flavonoids extract of Desmodium Styracifolium being of a granularity ranging from 50 to 100 meshes. A content of the total flavonoids of Desmodium Styracifolium in the extract reaches 50% to 80%, in which a content of schaftoside (dried product, %) is from 3.0% to 12.0%. The extract after drying is collected, sealed, weighted and stored in a dry place.

Ethanol concentration described herein refers to a volume fraction (V/V) of ethanol contained in 100 mL ethanol-water solution.

Specifically, according to some embodiments of the present disclosure, the preparation process and the technology parameters for extracting the total flavonoids of Desmodium Styracifolium are investigated and studied in detail, resulting in a preferable condition, which is verified in a pilot test and successfully transited into industrial production.

Specifically, in an embodiment of the present disclosure, a method for preparing the total flavonoids extract of Desmodium Styracifolium may include the following steps:

weighing a raw material of Desmodium Styracifolium, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing;

concentrating the alcohol extracting solution to be of a volume 5 times the weight of the raw material followed by still standing and filtering, to obtain a filtrate;

subjecting the filtrate to adsorption onto an AB-8 macroporous resin column at a flow rate of 3 column bed volumes per hour, eluting and purifying with water having a volume 10 times the weight of filled resin, and eluting with ethanol having a concentration of 60% and a volume of 8 column bed volumes at a flow rate of 3 column bed volumes per hour, to obtain an eluted solution; and

concentrating the eluted solution to recycle ethanol and to obtain a concentrated solution with a relative density of 1.22 followed by drying under reduced pressure at a temperature of 75° C. and then smashing to obtain the total flavonoids extract of Desmodium Styracifolium in a form of fine powder with a granularity ranging from 50 to 100 meshes.

Contents of effective ingredients and effective substances in Desmodium Styracifolium drugs have been increased according to present disclosure. A content of total flavonoids of Desmodium Styracifolium is between 50% and 80% (by the extract after dried, %), in which a content of schaftoside is between 3.0% and 12.0% (by the extract after dried, %).

In an embodiment of the present disclosure, the pharmaceutical composition consists of total flavonoids extract of Desmodium Styracifolium and a pharmaceutically acceptable excipient, so as to enable the pharmaceutical composition to present in a form of pharmaceutical preparations suitable for clinical administration. Alternatively, the pharmaceutical composition of the present disclosure can be present in at least one oral formulation form selecting from tablets, effervescent capsules, hard capsules, soft capsules, granules, electuary, pills, or powders, wherein the tablets may be sugar-coated tablets, film-coated tablets, enteric-coated tablets, dispersible tablets, sustained-release tablets, controlled-release tablets, or effervescent tablets. Therefore, the pharmaceutical composition is suitable for administration to a subject.

In a second aspect, there is provided use of the pharmaceutical composition described above in preparation of a medicament, in which the pharmaceutical composition is used to treat urinary stone, that is, the pharmaceutical composition of the present disclosure can be effectively used in scavenging dampness-heat, expelling stone through diuresis, alleviating a dribbling pain caused by stagnation of dampness-heat and urinary stone. The pharmaceutical composition may be used in preparation of a clinical therapeutic medicament for scavenging dampness-heat or expelling stone through diuresis (stagnation of dampness-heat).

Based on general pharmacological experiments performed according to embodiments of the present disclosure, after administration of the total flavonoids of Desmodium Styracifolium, there is no obvious change in behaviour, reaction, action, emotion and gait of the animal, and there is no effect on spontaneous activity of the animal, on excitability to central nervous system of the animal or on gastrointestinal movement of the mouse. The results from pharmacological experiments according to embodiments of the present disclosure show that: the total flavonoids of Desmodium Styracifolium may obviously inhibit an amount of calcium oxalate crystalline polymer in kidney, and decrease formation rate of kidney stone and reduce content of creatinine and uric acid, thus improving the kidney function of rat. The total flavonoids of Desmodium Styracifolium may have functions in dissolving stones and reducing formation of new stones, and may also have diuretic effect. Moreover, the total flavonoids of Desmodium Styracifolium may reduce welling degree and swelling rate caused by injecting egg albumen to toes of rats, which indicates that the total flavonoids of Desmodium Styracifolium may have certain anti-inflammatory effect and have obvious inhibiting effect on proliferation of granulation tissue. Moreover, acute toxicity tests were performed to animals for evaluating safety of the total flavonoids of Desmodium Styracifolium. Mice were administrated with the total flavonoids of Desmodium Styracifolium by gavage for acute toxicity observation, corresponding results show that the total flavonoids of Desmodium Styracifolium are substantially nontoxic to the mice. Rats were administrated with the total flavonoids of Desmodium Styracifolium by gavage for acute toxicity observation, corresponding results show that there is no server acute toxicity for the rats administrated with the total flavonoids of Desmodium Styracifolium. In long term toxicity tests, the total flavonoids of Desmodium Styracifolium have also been proven to be safe for animals. In an embodiment of the present disclosure, the medicament can present in at least one oral formulation form selected form tablets, effervescent capsules, hard capsules, soft capsules, granules, electuary, pills, or powders, wherein the tablets may be sugar-coated tablets, film-coated tablets, enteric-coated tablets, dispersible tablets, sustained-release tablets, controlled-release tablets, or effervescent tablets.

In a third aspect, a method for preparing a pharmaceutical composition is provided. In an embodiment of the present disclosure, the method includes: providing alcohol extract of Desmodium Styracifolium containing total flavonoids of Desmodium Styracifolium as an active ingredient. In some embodiments of the present disclosure, the method further includes a step of adding a pharmaceutically acceptable excipient, wherein the total flavonoids of Desmodium Styracifolium is of a content ranging from 2.5 wt % to 95 wt % based on a total weight of the pharmaceutical composition. The total flavonoids extract of Desmodium Styracifolium is prepared by: extracting a raw material of Desmodium Styracifolium with ethanol having a concentration ranging from 50% to 95% and a weight ranging from 8 to 14 times as heavy as the raw material, so as to obtain an extracting solution of Desmodium Styracifolium, and purifying the extracting solution of Desmodium Styracifolium, so as to obtain the total flavonoids extract of Desmodium Styracifolium; concentrating the extracting solution of Desmodium Styracifolium, so as to remove ethanol; and subjecting the extracting solution of Desmodium Styracifolium after concentrated to adsorption onto a macroporous resin column, so as to obtain the alcohol extract of Desmodium Styracifolium. In an embodiment of the present disclosure, resulting total flavonoids extract of Desmodium Styracifolium can be dried to obtain a solid product as required.

The urinary stone disease can be effectively treated with the pharmaceutical composition obtained by above method.

In an embodiment of the present disclosure, extracting the raw material of Desmodium Styracifolium with ethanol further includes: heating and refluxing the raw material of Desmodium Styracifolium for extraction, 1 to 3 times with 1 to 3 hours for each time, with ethanol having a concentration ranging from 50% to 95% and a weight ranging from 8 to 14 times as heavy as the raw material of Desmodium styracifolium, to obtain alcohol extracting solutions of Desmodium styracifolium followed by mixing, so as to obtain the extracting solution of Desmodium styracifolium. In a specific embodiment of the present disclosure, purifying the extracting solution of Desmodium Styracifolium further includes: concentrating the extracting solution of Desmodium Styracifolium, so as to remove ethanol; and subjecting the extracting solution of Desmodium Styracifolium after concentrated to adsorption onto a macroporous resin column, so as to obtain purified total flavonoids extract of Desmodium Styracifolium.

In specific, in an embodiment of the present disclosure, a method for preparing the total flavonoids extract of Desmodium Styracifolium may include: heating and refluxing a raw material of Desmodium Styracifolium for extraction, 1 to 3 times with 1 to 3 hours for each time, at a temperature of 50° C. to 60° C. with ethanol having a concentration of 50% to 95% and a weight ranging from 8 to 14 times as heavy as the raw material, so as to obtain alcohol extracting solutions of Desmodium Styracifolium followed by mixing; concentrating the alcohol extracting solution to be of a volume 2 to 8 times the weight of the raw material followed by still standing and filtering to obtain a filtrate; subjecting the filtrate to adsorption onto an AB-8 macroporous resin column at a flow rate ranging from 1 to 3 column bed volumes per hour; eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and concentrating the eluted solution to recycle ethanol and to obtain a concentrated solution with a relative density ranging from 1.10 to 1.30 followed by drying and then smashing so as to obtain fine powder of the total flavonoids extract of Desmodium Styracifolium.

In a specific embodiment of the present disclosure, alternatively, a method for preparing the extract of Desmodium Styracifolium is as follow: weighing 50 g raw material of Desmodium Styracifolium, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material for the second time, so as to obtain alcohol extracting solutions of Desmodium Styracifolium followed by mixing; concentrating the alcohol extracting solution to be of a volume 5 times the weight of the raw material followed by still standing and filtering, to obtain a filtrate (a loading sample) for use. 100 g AB-8 macroporous resin (in pharmaceutical grade) is immersed into a suitable amount of ethanol, and then packed into a column by a wet method followed by treatment for use. The filtrate (the loading sample) is subjected to adsorption onto an AB-8 macroporous resin column at a flow rate of 2 column bed volumes per hour, eluted and purified with water having a volume 10 times the weight of filled resin, and eluting with ethanol having a concentration of 60% and a volume of 8 column bed volumes at a flow rate of 2 column bed volumes per hour, to obtain an eluted solution; concentrating the eluted solution to recycle ethanol and to obtain a concentrated solution with a relative density of 1.22 followed by drying under reduced pressure at a temperature of 75° C. and then smashing, thereby obtaining 1.10 g total flavonoids extract of Desmodium Styracifolium.

In an embodiment of the present disclosure, a method for preparing a medicament may further includes a step of formulating into a traditional Chinese preparation by adding a pharmaceutically acceptable excipient. In an embodiment of the present disclosure, a method for preparing a medicament may further includes the following steps: formulating the medicament into at least one oral formulation form selecting from tablets, effervescent capsules, hard capsules, soft capsules, granules, electuary, pills, or powders, wherein the tablets may be sugar-coated tablets, film-coated tablets, enteric-coated tablets, dispersible tablets, sustained-release tablets, controlled-release tablets, or effervescent tablets.

In specific, a method for preparing a pharmaceutical composition in the form of the granules may include: dissolving an adhesion agent in a formula dosage into water under stirring to be uniform, so as to prepare an adhesion agent solution for use; mixing the total flavonoids extract of Desmodium Styracifolium in a formula dosage and a filler in a fluidized bed; granulating by spraying, with a gunjet, an adhesion agent solution into the fluidized bed; drying and discharging resulting particles to obtain mixed power; and packaging the mixed power so as to obtain the pharmaceutical composition in the form of the granules.

In specific, a method for preparing a pharmaceutical composition in the form of the granules may further include: sieving the total flavonoids extract of Desmodium Styracifolium and the pharmaceutically acceptable excipient in respective formula dosage at 60 to 100 meshes; dissolving an adhesion agent into a solvent under stirring to obtain an adhesion agent solution for use; preheating the alcohol extract of Desmodium Styracifolium and the pharmaceutically acceptable excipient, sieved in advance, to a temperature of 35° C. to 55° C. within 5 min to 60 min in a fluidized bed; granulating by spraying, with a gunjet under an atomizing pressure ranging from 0.7 Bar to 1.0 Bar (1 Bar=0.1 MPa) and a spraying speed ranging from 15 rpm/min to 25 rpm/min, the adhesion agent solution into the fluidized bed adjusted with an air inlet temperature of 50° C. to 65° C. to enable materials therein to be of a material temperature between 40° C. to 55° C., by which the adhesion agent is completely sprayed within 5 min to 60 min; drying resulting particles in the fluidized bed adjusted with the air inlet temperature of 60° C. to 70° C. to enable materials therein to be of the material temperature of 40° C. to 55° C. for 5 min to 60 min; cooling and discharging the particles to obtain mixed power; and packaging the mixed power so as to obtain the pharmaceutical composition in the form of the granules.

In a specific embodiment of the present disclosure, a method for preparing the granules containing the pharmaceutical composition of the present disclosure is as follow: dissolving 1 g adhesion agent such as povidone K₃₀ into 120 g water under stirring to be uniform, so as to obtain an adhesion agent solution for use; preheating 133 g total flavonoids extract of Desmodium Styracifolium, 110 g microcrystalline cellulose and 60 g lactose, mixed in advance, to a temperature of 45° C. within 20 min in a fluidized bed; granulating by spraying, with a gunjet under an atomizing pressure of 0.9 Bar and a spraying speed of 20 rpm/min, the adhesion agent solution into the fluidized bed adjusted with an air inlet temperature of 55° C. to enable materials therein to be of a material temperature of 45° C., by which the adhesion agent solution is completely sprayed within 15 min; drying resulting particles in the fluidized bed adjusted with the air inlet temperature of 65° C. to enable materials therein to be of the material temperature of 45° C. for 10 min; cooling and discharging the particles to obtain mixed power; and packaging the mixed power so as to obtain 1000 packages of the pharmaceutical composition in the form of the granules.

In specific, a method for preparing a pharmaceutical composition in the form of hard capsules may include: dissolving an adhesion agent in a formula dosage into water under stirring to be uniform, so as to obtain an adhesion agent solution for use; granulating by spraying, with a gunjet, an adhesion agent solution towards the alcohol extract of Desmodium Styracifolium and a filler mixed in advance in a fluidized bed, thereby obtaining particles; drying and discharging the particles to obtain mixed power; and capsulizing the mixed power with an encapsulating machine, so as to obtain the pharmaceutical composition in the form of the capsules.

In specific, a method for preparing a pharmaceutical composition in the form of the hard capsules may further include: sieving the total flavonoids extract of Desmodium Styracifolium and a pharmaceutically acceptable excipient in respective formula dosage at 60 to 100 meshes; dissolving an adhesion agent into a solvent under stirring to obtain an adhesion agent solution for use; preheating the total flavonoids extract of Desmodium Styracifolium and the pharmaceutically acceptable excipient in the respective formula dosage, sieved in advance, to a temperature of 35° C. to 55° C. within 5 min to 60 in a fluidized bed; granulating by spraying, with a gunjet under an atomizing pressure ranging from 0.7 Bar to 0.1 Bar (1 Bar=0.1 MPa) and a spraying speed ranging from 15 rpm/min to 25 rpm/min, an adhesion agent into the fluidized bed adjusted with an air inlet temperature of 50° C. to 65° C. to enable materials therein to be of a material temperature between 40° C. to 55° C., by which the adhesion agent is completely sprayed within 5 min to 60 min; drying resulting particles in the fluidized bed adjusted with an air inlet temperature of 60° C. to 70° C. to enable materials therein to be of a material temperature of 40° C. to 55° C. for 5 min to 60 min; cooling and discharging the particles to obtain mixed power; and capsulizing the mixed power, so as to obtain the pharmaceutical composition in the form of the capsules.

In a specific embodiment of the present disclosure, a method for preparing the capsules containing the total flavonoids extract of Desmodium Styracifolium is as follow: dissolving 1 g adhesion agent such as povidone K₃₀ into 120 g water under stirring to be uniform, so as to obtain an adhesion agent solution for use; preheating 133 g total flavonoids extract of Desmodium Styracifolium, 30 g microcrystalline cellulose and 37 g lactose, mixed in advance, to a temperature of 45° C. within 20 min in a fluidized bed; granulating by spraying, with a gunjet under an atomizing pressure of 0.09 MPa and a spraying speed of 20 rpm/min, the adhesion agent solution into the fluidized bed adjusted with an air inlet temperature of 55° C. to enable materials therein to be of a material temperature of 45° C., by which the adhesion agent solution is completely sprayed within 15 min; drying resulting particles in the fluidized bed adjusted with the air inlet temperature of 65° C. to enable materials therein to be of the material temperature of 45° C. for 10 min; cooling and discharging the particles to obtain mixed power, and capsulizing the mixed power with an encapsulating machine, so as to obtain 1000 capsules of the pharmaceutical composition in the form of the capsules.

In specific, a method for preparing a pharmaceutical composition in the form of the tablets may include: dissolving the total flavonoids extract of Desmodium Styracifolium, a solid dispersion carrier and a surfactant in respective formula dosage, sieved in advance respectively, with 50% ethanol under heating and stirring, followed by removing the solvent via evaporation under reduced pressure, vacuum drying, smashing and sieving, so as to obtain solid dispersion containing total flavonoids of Desmodium Styracifolium; mixing with a filler and a disintegrant, sieved in advance respectively, to be uniform, followed by granulating, drying, size stabilizing, and mixing with a lubricant to be uniform, so as to obtain particles of the total flavonoids of Desmodium Styracifolium; tableting the particles of the total flavonoids of Desmodium Styracifolium with a tableting machine so as to obtain the pharmaceutical composition in the form of tablets. Further, the tablets may be coated with sugar or film, so as to obtain the pharmaceutical composition in the form of sugar-coated tablets or film-coated tablet forms.

In specific, a method for preparing a pharmaceutical composition in the form of tablets may further include: dissolving the total flavonoids of Desmodium Styracifolium, a solid dispersion carrier and a surfactant in respective formula dosage, sieved at 40 to 200 meshes in advance respectively, with 50% ethanol under stirring and heating at a temperature of 50° C. to 75° C., followed by removing the solvent via evaporation under reduced pressure at a temperature of 30° C. to 75° C., vacuum drying at a temperature of 30° C. to 60° C., smashing and sieving at 40 to 200 meshes, so as to obtain solid dispersion containing total flavonoids of Desmodium Styracifolium for use; mixing a filler and a disintegrant, sieved at 40 to 100 meshes in advance, respectively, with the solid dispersion containing total flavonoids of Desmodium Styracifolium to be uniform, preparing a soft material, granulating at 10 to 30 meshes, drying at a temperature of 30° C. to 75° C., size stabilizing, and then mixing with a lubricant to be uniform, thereby obtaining the particles containing total flavonoids of Desmodium Styracifolium; tableting the particles containing total flavonoids of Desmodium Styracifolium with a tableting machine, so as to obtain the pharmaceutical composition in the forms of tablets. Further, the tablets may be coated with sugar or film, so as to obtain the pharmaceutical composition in the form of sugar-coated tablets or film-coated tablets.

In a specific embodiment of the present disclosure, a method for preparing the tablets containing the total flavonoids of Desmodium Styracifolium is as follow: dissolving 66.5 g total flavonoids of Desmodium Styracifolium, 266 g povidone K₃₀, 133 g poloxamer 188, and 39.9 g sodium dodecyl sulfate in the respective formula dosage, sieved at 80 meshes in advance respectively, with 50% ethanol under stirring and heating at a temperature of 65° C., followed by removing the solvent via evaporation under reduced pressure at a temperature of 50° C., vacuum drying at a temperature of 40° C., smashing and sieving at 80 meshes, so as to obtain solid dispersion containing the total flavonoids of Desmodium Styracifolium for use; mixing 10 g lactose and 15 g sodium croscarmellose, sieved at 80 meshes in advance respectively, with the solid dispersion containing the total flavonoids of Desmodium Styracifolium to be uniform, preparing a soft material with proper water, granulating at 20 meshes, drying at a temperature of 55° C., size stabilizing, and then mixing with 6 g sodium stearyl fumarate to be uniform, thereby obtaining the particles containing total flavonoids of Desmodium Styracifolium; tableting the particles containing total flavonoids of Desmodium Styracifolium with a tableting machine, so as to obtain 1000 tablets of the pharmaceutical composition in the forms of tablets. Further, the tablets may be coated with sugar or film, so as to obtain the pharmaceutical composition in the form of sugar-coated tablets or film-coated tablets.

In specific, a method for preparing the pharmaceutical composition in the form of the soft capsules may include: mixing an oil phase, a surfactant and a co-surfactant in respective formula dosage to be uniform under stirring or ultrasonic treatment to obtain a mixture; dissolving the total flavonoids extract of Desmodium Styracifolium in its formula dosage into the mixture under stirring or ultrasonic treatment, and capsulizing into a soft capsule to obtain the pharmaceutical composition in the form of the soft capsules.

In specific, a method for preparing the pharmaceutical composition in the form of the soft capsules may further include: mixing 40 g soybean oil, 80 g polyoxyethylene (40) hydrogenated castor oil and 30 g polyethylene glycol 400 in respective formula dosage under stirring or ultrasonic treatment to be uniform, so as to obtain a mixture; dissolving 133 g total flavonoids extract of Desmodium Styracifolium in its formula dosage into the mixture under stirring or ultrasonic treatment at a temperature of 37° C., thereby obtaining content fluids after degassing under vacuum; and filling and compressing content fluids in a soft capsule pelleting machine, thereby obtaining soft capsules; hardening and molding by blow-drying in drum drying equipment, followed by sterilizing and scrubbing with ethanol, natural evaporating the moisture in capsule shell and ethanol for 20 h such that capsule shell is of a dried, smooth and slightly elastic surface, which are subjected to selection for discarding unqualified soft capsules with unqualified appearance and seam and packing qualified soft capsule in a bottle or blister plate, thereby obtaining 1000 capsules containing the pharmaceutical composition in the form of the soft capsules.

In an embodiment of the present disclosure, a medicament prepared by the present method can be used in the treatment of urinary stone.

In a fourth aspect, there is provided a medicament prepared by above method. As described before, urinary stone can be effectively treated by using the medicament.

It should be noted that, the pharmaceutical composition, the method for preparing the same and use thereof according to the present disclosure are accomplished through hard creative labour and optimization work by inventors of the present application.

As compared with the related art, the technical solution of the present disclosure has advantages as described below.

1. According to embodiments of the present disclosure, in the process for extracting and purifying the raw material, ethanol is used as an extraction solvent for extracting the raw material of Desmodium Styracifolium, and the extracting solution is purified by a macroporous adsorption resin to obtain the active ingredient of Desmodium Styracifolium, i.e., the total flavonoids of Desmodium Styracifolium. As compared with a water-extraction and alcohol-precipitation method for extracting, the active ingredient basis of the extract by such a process is clear and the quality standard is controllable, thus decreasing the dosage for clinical administration and reducing clinical side effects.

2. As compared with a process of ethanol extracting and macroporous resin purifying in the related art, ethanol is recycled from the extracting solution in embodiments of the present disclosure, so that the extracting solution is concentrated to be of a certain volume (5 times of the weight of the raw material) as a consequence, which can be directed purified by the macroporous resin without special concentrating and drying for into extractum, thus saving time for preparation. Besides, after purified by the macroporous resin, the active ingredient with a high content is eluted even using ethanol with the same concentration, which is a simple process and has a good operability as compared with gradient dilution using ethanol with different concentrations. Thirdly, the total flavonoids of Desmodium Styracifolium (i.e., the active ingredient of Desmodium Styracifolium) is obtained by recycling ethanol from the eluted solution and directly drying under reduced pressure without any solvent for processing, thus saving consumption in the preparation. In the view of scale production, the above mentioned process for extracting and purifying decreases production costs, shortens production period, which is simple, convenient and practical, thus meeting requirements to modern industry of Chinese medicine.

3. According to embodiments of the present disclosure, the method includes extracting and purifying the active ingredients by means of AB-8 macroporous adsorption resin technique, which is a simple process with low costs as the resin, is reusable, thus being suitable for industry production. Moreover, in embodiments of the present disclosure, an optimal condition has been selected by carefully investigating corresponding parameters, which is verified in a pilot test and can be transited into industrial production, thereby increasing the content of the active ingredient.

4. As compared with commercially available medicaments with the same use, the pharmaceutical composition containing total flavonoids of Desmodium Styracifolium prepared according to embodiments of the present disclosure has a developed production process, a clear active ingredient basis, a controllable quality, a clear and definite clinical indication, a significant pharmacological efficacy, a small dosage, a safe and convenient administration and a mild side effect, so that the pharmaceutical composition of the present disclosure has the advantage of being suitable to technique and quality standard of the modern manufacturing industry.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 is a leaking curve showing an amount of total flavonoids of Desmodium Styracifolium in a sample solution adsorbed by an AB-8 macroporous resin column according to an embodiment of the present disclosure; and

FIG. 2 is an eluting curve showing an amount of total flavonoids of Desmodium Styracifolium eluted by the resin column with 60% ethanol as an eluent according to an embodiment of the present disclosure.

### DETAILED DESCRIPTION

In the following, embodiments of the present disclosure will be described in detail, whose examples will be shown in drawings. The same or similar elements and the elements having same or similar functions are denoted by like reference numerals throughout the descriptions. Embodiments described in the following with reference to drawings are explanatory, illustrative, and used to generally understand the present disclosure, and shall not be construed to limit the present disclosure.

### Embodiment 1

### Preparation of Total Flavonoids of Desmodium Styracifolium

(1) Extracting method: free flavonoids and flavonoid glycosides generally can be extracted with an organic solvent according to the solubility of flavonoids, for example, ethanol with relative high concentration is commonly used in industrial production. Based on common industrialized method for extracting flavonoids, the extraction in the present study is performed with ethanol having a concentration of 60% to 95% (as an extraction solvent) twice, which is more economical and practical according to conventional production method.

Study on extraction process of refluxing with ethanol: Parameters for the extraction process, including ethanol concentration, ethanol weight (with respect to the weight of raw material of Desmodium Styracifolium), and extraction period, were determined by L9 (34) orthogonal tests, which took the total flavonoids of Desmodium Styracifolium as index. A content of the total flavonoids in an extract is measured by UV-visible spectrophotometry, and comparative analysis was performed with the content of total flavonoids and a net weight of total flavonoids in a dry extract as evaluation indexes. Factors and levels of experiments were designed as shown in Table 1, and analysis results are shown in Table 2.

TABLE 1
factors and levels of experiments

| | | B | | | | |
|---|---|---|---|---|---|---|
| | | Ethanol amount (with | | | | |
| | A | respect to the weight of raw | | | | |
| | Ethanol | material of Desmodium | | | D | |
| | concentration | Styracifolium) | | C | extraction period (h) | |
| Levels\Factors | (%) | 1^{st} time | 2^{nd} time | Control | 1^{st} time | 2^{nd} time |
| 1 | 60 | 8 | 6 | — | 1.0 | 1.0 |
| 2 | 80 | 10 | 8 | — | 1.5 | 1.0 |
| 3 | 95 | 12 | 10 | — | 2.0 | 1.5 |

TABLE 2
orthogonal tests and analysis results

| | | | | | Content | Net weight |
|---|---|---|---|---|---|---|
| Experi- | | | | | of total | of total |
| ment | | | | | flavonoids | flavonoids |
| No. | A | B | C | D | (%) | (g) |
| 1 | 1 | 1 | 1 | 1 | 19.0 | 0.67 |
| 2 | 1 | 2 | 2 | 2 | 23.8 | 1.01 |
| 3 | 1 | 3 | 3 | 3 | 23.0 | 1.11 |
| 4 | 2 | 1 | 2 | 3 | 27.4 | 1.05 |
| 5 | 2 | 2 | 3 | 1 | 25.5 | 1.03 |
| 6 | 2 | 3 | 1 | 2 | 30.7 | 1.15 |
| 7 | 3 | 1 | 3 | 2 | 15.0 | 0.45 |
| 8 | 3 | 2 | 1 | 3 | 16.0 | 0.48 |
| 9 | 3 | 3 | 2 | 1 | 16.8 | 0.49 |
| K1 | 2.790 | 2.170 | 2.300 | 2.190 | | |
| K2 | 3.230 | 2.520 | 2.550 | 2.610 | | |
| K3 | 1.420 | 2.750 | 2.590 | 2.640 | | |
| k1 | 0.930 | 0.723 | 0.767 | 0.730 | | |
| k2 | 1.077 | 0.840 | 0.850 | 0.870 | | |
| k3 | 0.473 | 0.917 | 0.863 | 0.880 | | |
| R | 0.604 | 0.194 | 0.096 | 0.150 | | |

Visual analysis: As can be seen from R values shown in Table 2, R_{A}>R_{B}>R_{D}, which shows that an order of factors is A>B>D. And as can be seen from k values, A2>A1>A3, B3>B2>B1, D3>D2>D1, therefore, an optimal level combination of factors is A2B3D3. That is, an optimum extraction process for total flavonoids of Desmodium Styracifolium is: extracting twice with 80% ethanol, having a weight of 12 times as heavy as the raw material of Desmodium styracifolium for 2 hours for the first time, and having a weight of 10 times as heavy as the raw material of Desmodium styracifolium for 1.5 hours for the second time.

(2) Study on purification process with an extracting solution of Desmodium Styracifolium obtained through above optimized extraction conditions by adsorption onto a macroporous resin column:

1) Study on Screening Macroporous Resins

Resin: AB-8 resin (Nankai University), D101 resin (Shandong Lukang), HPD100 resin (Hebei Baocang Co., Ltd).

a) Studies on Static Saturated Adsorption and Desorption Elution with Total Flavonoids in the Sample Solution Using Different Macroporous Adsorption Resins

2 g well-treated (pumping filtrated till without water drop) macroporous resin was weighted precisely, and added into 100 mL ground erlenmeyer flask, followed by precisely added with 50 mL sample solution and then continuously shaken 24 hours on an oscillator for fully adsorption. The resulting supernatants was measured with the concentrations of total flavonoids therein. The saturated adsorption capacity of the resin was calculated by the following formula: saturated adsorption capacity=[(initial concentration−concentration after adsorption)×volume of adsorption solution]/resin weight], elution rate=(eluent concentration×elution volume)/saturated adsorption capacity×100%, and the results are shown in Table 3.

Table 3 results of static saturated adsorption and desorption as for three types of resin

Total flavonoids

| | Saturated adsorption | Elution rate |
|---|---|---|
| Resin type | capacity (mg/g) | (%) |
| AB-8 | 58.36 | 89.75 |
| D101 | 49.27 | 81.23 |
| HPD100 | 52.41 | 85.35 |

The results show that, saturated adsorption capacities, elution volumes and elution rates of AB-8, D101, HPD100 types of macroporous resins for total flavonoids in the sample solution are relatively close in the static adsorption and desorption tests, and static adsorption and desorption properties of the three types of macroporous resins were further investigated.

b) Study on Static Saturated Adsorption and Desorption Properties with Total Flavonoids in the Sample Solution Using the Three Types of Macroporous Adsorption Resins

For three types of macroporous resins, 2 g each type of macroporous resins was weighed precisely and packed into a column for use. 100 mL loading sample was subjected to adsorption onto respective macroporous resin column at a flow rate of 1 mL/min thereby obtaining first effluents, which was subjected to adsorption again with the same column thereby obtaining second effluents. Each macroporous resin column adsorbed with loading sample was eluted with a certain volume of water to obtain an eluted solution, which was used for measuring the content of total flavonoids in the eluted solution. The adsorption ratio of respective macroporous resin was calculated based on the following formula: adsorption ratio=[(a substance content in the loading sample−a substance content in the second effluent−a substance content in the eluted solution)]/filled resin weight], elution ratio=[concentration of the eluted solution×volume of adsorbed solution/filled resin weight], and the results are shown in Table 4.

Table 4 results of saturated adsorption and desorption of three types of resin

Total flavonoids

| Resin type | Adsorption ratio (mg/g) | Elution ratio (%) |
|---|---|---|
| AB-8 | 50.24 | 86.52 |
| D101 | 40.78 | 78.34 |
| HPD100 | 45.02 | 80.19 |

The results show that, the AB-8 macroporous resin is of good adsorption ratio and the elution ratio of total flavonoids of Desmodium Styracifolium, and high safety, which has been most applied in domestic pharmaceutical manufacturing industry, therefore, the present study selected AB-8 macroporous resin to purify the total flavonoids of Desmodium Styracifolium.

2) Study on Purification Process with AB-8 Macroporous Resin

a) Optimization to Adsorption Conditions

L9 (34) orthogonal table was used to design levels and factors including a concentration of the loading sample (by the weight of raw material contained in the loading sample), an adsorption velocity and a ratio of diameter to height, as shown in Table 5. Contents of the total flavonoids were measured for the following 9 testing groups, adsorption ratios were calculated, and comprehensive evaluation was performed. Analysis results are shown in Table 6.

TABLE 5
Table of factors and levels

| | Factors | | | |
|---|---|---|---|---|
| | A | B | | D |
| | concentration of | adsorption velocity | | ratio of |
| | loading sample | (multiples of column | C | diameter |
| Levels | (g/mL) | volume/h) | Control | to height |
| 1 | 0.1 | 2 | — | 1:4 |
| 2 | 0.2 | 4 | — | 1:8 |
| 3 | 0.4 | 6 | — | 1:12 |

TABLE 6
orthogonal design and analysis results

| | | | | | Content | Net weight |
|---|---|---|---|---|---|---|
| Experi- | | | | | of total | of total |
| ment | | | | | flavonoids | flavonoids |
| NO. | A | B | C | D | (%) | (g) |
| 1 | 1 | 1 | 1 | 1 | 70.35 | 0.28 |
| 2 | 1 | 2 | 2 | 2 | 66.75 | 0.22 |
| 3 | 1 | 3 | 3 | 3 | 63.08 | 0.26 |
| 4 | 2 | 1 | 2 | 3 | 69.21 | 0.25 |
| 5 | 2 | 2 | 3 | 1 | 66.06 | 0.24 |
| 6 | 2 | 3 | 1 | 2 | 68.54 | 0.28 |
| 7 | 3 | 1 | 3 | 2 | 60.97 | 0.27 |
| 8 | 3 | 2 | 1 | 3 | 61.21 | 0.24 |
| 9 | 3 | 3 | 2 | 1 | 66.09 | 0.20 |
| K1 | 0.760 | 0.800 | 0.800 | 0.720 | | |
| K2 | 0.770 | 0.700 | 0.670 | 0.770 | | |
| K3 | 0.710 | 0.740 | 0.770 | 0.750 | | |
| k1 | 0.253 | 0.267 | 0.267 | 0.240 | | |
| k2 | 0.257 | 0.233 | 0.223 | 0.257 | | |
| k3 | 0.237 | 0.247 | 0.257 | 0.250 | | |
| R | 0.020 | 0.034 | 0.044 | 0.017 | | |

Result analysis: As can be seen from R values shown in Table 2, RB>RA>RD, which shows that an order of factors was B>A>D. And as can be seen from k values, A2>A1>A3, B1>B2>B2, D2>D3>D1, therefore, an optimal level combination of factors was A2B13D2. Therefore, an optimum adsorption condition of total flavonoids preferably is: 0.2 g/ml of loading sample, 2 BV/h of adsorption velocity, and 1:8 of ratio of diameter to height.

b) Investigation in a Volume of Loading Sample

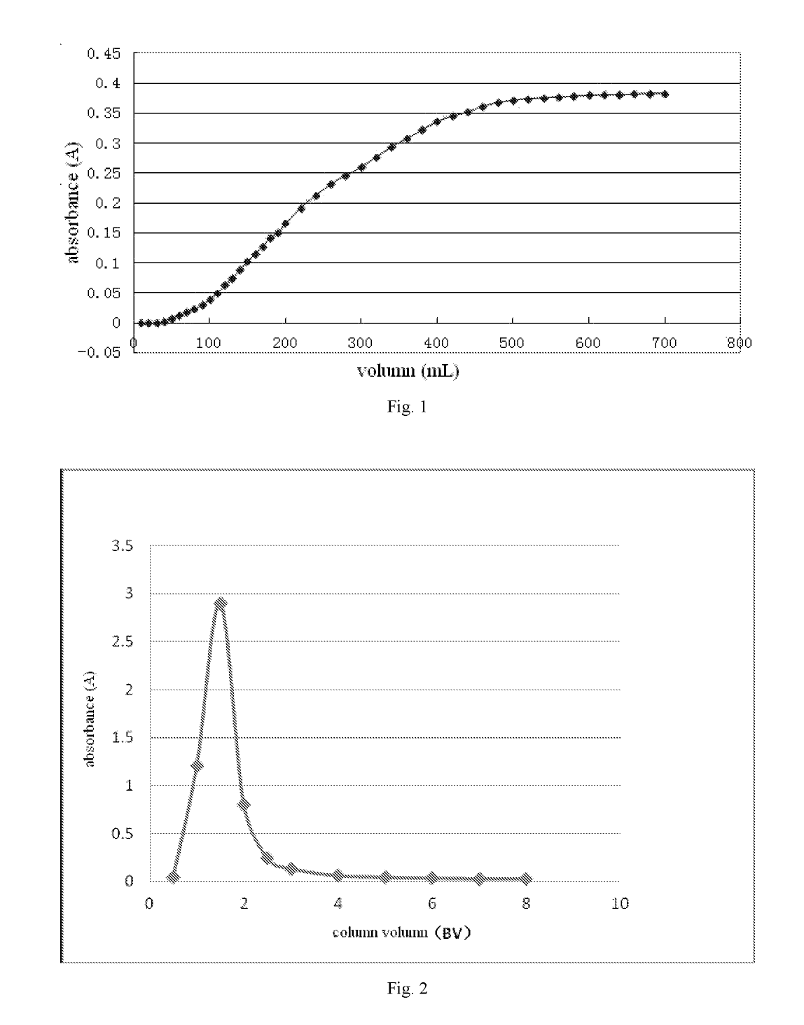

0.2 g/mL loading sample was applied onto 20 g AB-8 resin column (400 mm×20 mm) at the flow rate of 2 Bv/h. 10 mL was collected from each fraction. Concentrations of total flavonoids in each fraction were measured and calculated, and a leaking curve was plotted, the results are shown in FIG. 1. As can be seen from the figure, total flavonoids begin to leak when the volume of the loading sample was 50 mL (i.e. 10 g raw material of Desmodium Styracifolium), and an adsorption saturation state was reached when the volume of the loading sample was 600 mL (about 30 times the weight of filled resin).

c) Investigation in Washing Condition

50 mL loading sample was subjected to adsorption in accordance above optimum adsorption condition, purified with water, with every 20 mL of effluent for one fraction, inspection was performed through α-naphthol reaction, at the same time, weight of dry extract was determined, α-naphthol reaction was negative and the weight of dry extract does not change anymore after washing with 300 mL water, indicating that sugars adsorbed by the resin column can be substantially removed after washing with 300 mL water (about 15 times the weight of resin).

d) Investigation in Ethanol Concentration for Elution

Another five copies of resin (with 20 g for each copy) each were subjected to adsorptions, purified in accordance above adsorption condition and washing condition followed by separately eluted with 400 mL ethanol each having a concentration of 30%, 45%, 60%, 75%, 90% at same flow rate, the content and desorption rate of total flavonoids were determined and calculated, results are shown in Table 7.

Table 7 results of ethanol concentration for elution of total flavonoids of Desmodium Styracifolium

| Ethanol concentration (%) | 30 | 45 | 60 | 75 | 90 |
|---|---|---|---|---|---|
| Desorption rate (%) | 38.16 | 59.25 | 85.63 | 81.56 | 79.21 |
| Weight of dry extract (mg) | 87.23 | 178.5 | 231.6 | 216.2 | 209.3 |
| Content of total | 35.21 | 42.06 | 58.63 | 55.45 | 56.82 |
| flavonoids (%) | | | | | |

The above results show that, both the desorption rate and content of total flavonoids are high when the ethanol concentration is 60% or more, and desorption capacities are considerable when the ethanol concentrations separately are 60%, 75% and 90%, the present test chose 60% ethanol as the elution solvent for the sake of manufacturing cost.

e) Investigation in Elution Velocity

Dynamic adsorption was performed with 60% ethanol as the elution solvent at flow rate of 1, 3, 5 column bed volumes per hour, respectively, according to above conditions, ethanol eluents were collected, the content and desorption rate of total flavonoids were determined and calculated, results are shown in Table 8.

Table 8 results of elution velocity of ethanol for elution of total flavonoids of Desmodium Styracifolium

| | | Content of total flavonoids in |
|---|---|---|
| Elution velocity | Desorption rate (%) | dry extract (%) |
| 1BV/h | 88.31 | 57.92 |
| 3BV/h | 87.45 | 56.37 |
| 5BV/h | 80.16 | 50.08 |

Results show that, there was no big difference between elution velocity of 1 column bed volume per hour and elution velocity of 3 column bed volumes per hour, and choosing elution velocity of 3 BV/h was more reasonable, considering the production efficiency.

f) Investigation in Elution Volume

Dynamic adsorption was performed with 60% ethanol as elution solvent according to above conditions, the effluent was quantitative collected and in which the content of total flavonoids were determined. Results are shown in FIG. 2. Results show that, flavonoids adsorbed by 20 g resin can be completely eluted with 240 mL (8 times the resin column volume) of 60% ethanol. Therefore, flavonoids adsorbed by 20 g resin can be completely eluted with 240 mL (8 times the resin column volume) of 60% ethanol at 3 BV/h of elution velocity.

### Embodiment 2

### Preparation of Total Flavonoids of Desmodium Styracifolium

50 g raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 g AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.10 g total flavonoids extract of Desmodium Styracifolium.

### Embodiment 3

### Preparation of Total Flavonoids of Desmodium Styracifolium

200 g raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 400 g AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 4.03 g total flavonoids extract of Desmodium Styracifolium (placed in a shady and cool place).

By means of UV-visible spectrophotometry, the content of the total flavonoids was measured to be of 63.31% (by dried product, %), the content of schaftoside was 5.38% (by dried product, %).

### Embodiment 4

### Preparation of Total Flavonoids of Desmodium Styracifolium

50 kg raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 kg AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.12 kg total flavonoids extract of Desmodium Styracifolium (placed in a shady and cool place).

By means of UV-visible spectrophotometry, the content of the total flavonoids was measured to be of 59.49% (by dried product, %), the content of schaftoside was 5.10% (by dried product, %).

### Embodiment 5

### Preparation of Total Flavonoids of Desmodium Styracifolium

50 kg raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 kg AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.14 kg total flavonoids extract of Desmodium Styracifolium (placed in a shady and cool place).

By means of UV-visible spectrophotometry, the content of the total flavonoids was measured to be of 59.37% (by dried product, %), the content of schaftoside was 5.01% (by dried product, %).

Results showed that, the process parameters studied by the present experiments was feasible, which can be successful transited to the industrial production after adjusted in pilot tests.

### Embodiment 6

### Preparation of Capsules of Total Flavonoids of Desmodium Styracifolium

### Formula

| | | |
|---|---|---|
| Total flavonoids of Desmodium Styracifolium | 133 | g |
| Lactose | 37 | g |
| microcrystalline cellulose | 30 | g |
| Povidone K₃₀ | 1 | g |
| Water | 120 | g |
| Total | 1000 | capsules |

Preparation was performed by the following steps:

a. preparing the total flavonoids extract of Desmodium Styracifolium according to the method in Embodiment 5;

b. dissolving 1 g adhesion agent such as povidone K₃₀ in 120 g water under stirring to be uniform, thereby obtaining an adhesion agent solution for use;

c. preheating 133 g total flavonoids extract of Desmodium Styracifolium, 30 g microcrystalline cellulose and 37 g lactose, mixed in advance, to a temperature of 45° C. for 20 min in a fluidized bed; granulating by spraying, with a gunjet under an atomizing pressure of 0.9 Bar and a spraying speed of 20 rpm/min, the adhesion agent solution into the fluidized bed adjusted with an air inlet temperature of 55° C. to enable the materials therein to be of a material temperature at 45° C., by which the adhesion agent solution is completely sprayed within 15 min;

d. drying resulting particles in the fluidized bed adjusted with the air inlet temperature of 65° C. to enable materials therein to be of the material temperature of 45° C. for 10 min;

e. cooling and discharging the particles to obtain mixed power; and capsulizing the mixed power with an encapsulating machine, so as to obtain the capsules containing total flavonoids of Desmodium Styracifolium.

### Embodiment 7

### Preparation of the Capsules of Total Flavonoids of Desmodium Styracifolium

### Formula

| | | |
|---|---|---|
| Total flavonoids of Desmodium Styracifolium | 133 | g |
| microcrystalline cellulose | 30 | g |
| Lactose | 37 | g |
| Povidone K₃₀ | 1 | g |
| Croscarmellose sodium | 20 | g |
| aerosil | 1 | g |
| Water | 120 | g |
| Total | 1000 | capsules |

### Preparation: Identical to Embodiment 6

### Embodiment 8

### Preparation of the Capsules of Total Flavonoids of Desmodium Styracifolium

### Formula

| | | |
|---|---|---|
| Total flavonoids of Desmodium Styracifolium | 100 | g |
| Lactose | 60 | g |
| sodium Croscarmellose | 35 | g |
| hydroxypropyl methyl cellulose | 0.1 | g |
| aerosil | 2 | g |
| Ethanol | 100 | g |
| Water | 10 | g |
| Total | 1000 | capsules |

### Preparation: Identical to Embodiment 6

### Embodiment 9

### Preparation of the Capsules of Total Flavonoids of Desmodium Styracifolium

### Formula

| | | |
|---|---|---|
| Total flavonoids of Desmodium Styracifolium | 66.5 | g |
| Lactose | 50 | g |
| Microcrystalline cellulose | 50 | g |
| Povidone K₃₀ | 1 | g |
| Water | 120 | g |
| Total | 1000 | capsules |

### Preparation: Identical to Embodiment 6

### Embodiment 10

### Preparation of the Granules of Total Flavonoids of Desmodium Styracifolium

### Formula

| | | |
|---|---|---|
| Total flavonoids of Desmodium Styracifolium | 133 | g |
| Lactose | 110 | g |
| Microcrystalline cellulose | 60 | g |
| Povidone K₃₀ | 1 | g |
| Water | 120 | g |
| Total | 1000 | bags |

Preparation was performed by the following steps:

a. preparing the total flavonoids extract of Desmodium Styracifolium according to the method in Embodiment 5;

b. dissolving 1 g adhesion agent such as povidone K₃₀ in 120 g water under stirring to be uniform, thereby obtaining an adhesion agent solution for use;

c. preheating 133 g total flavonoids extract of Desmodium Styracifolium, 60 g microcrystalline cellulose and 110 g lactose, mixed in advance, to a temperature of 45° C. for 20 min in a fluidized bed; granulating by spraying, with a gunjet under an atomizing pressure of 0.9 Bar and a spraying speed of 20 rpm/min, the adhesion agent solution into the fluidized bed adjusted with an air inlet temperature of 55° C. to enable the materials therein to be of a material temperature at 45° C., by which the adhesion agent solution is completely sprayed within 15 min;

d. drying resulting particles in the fluidized bed adjusted with the air inlet temperature of 65° C. to enable materials therein to be of the material temperature of 45° C. for 10 min;

e. cooling and discharging the particles to obtain mixed power; and packaging the mixed power, so as to obtain the granules containing total flavonoids of Desmodium Styracifolium.

### Embodiment 11

### Preparation of Granules of Total Flavonoids of Desmodium Styracifolium

### Formula

| | | |
|---|---|---|
| Total flavonoids of Desmodium Styracifolium | 150 | g |
| Microcrystalline cellulose | 60 | g |
| Lactose | 100 | g |
| Cross-linked povidone | 20 | g |
| Polyethylene glycol 6000 | 5 | g |
| Sodium stearyl fumarate | 5 | g |
| Ethanol | 100 | g |
| Water | 40 | g |
| Total | 1000 | bags |

### Preparation: Identical to Embodiment 10

### Embodiment 12

### Preparation of Tablets of Total Flavonoids of Desmodium Styracifolium

### Formula

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 39.9 | g |
| Lactose | 10 | g |
| sodium Croscarmellose | 15 | g |
| Sodium stearyl fumarate | 6 | g |
| Total | 1000 | tablets |

Preparation was performed by the following steps:

mixing 66.5 g the alcohol extract of Desmodium Styracifolium, 266 g povidone K₃₀, 133 g poloxamer 188 and 39.9 g sodium dodecyl sulfate, sieved at 80 meshes in advance respectively, with 50% ethanol, stirring and heating to a temperature of 65° C. for dissolution, removing the solvent via evaporation under reduced pressure at a temperature of 50° C., vacuum drying at a temperature of 40° C., and then smashing, so as to obtain the solid dispersion containing total flavonoids of Desmodium Styracifolium;

mixing 10 g lactose and 15 g sodium croscarmellose, sieved at 80 meshes in advance respectively, with the solid dispersion containing total flavonoids of Desmodium Styracifolium to be uniform, preparing a soft material, granulating at 20 meshes, drying at a temperature of 55° C., size stabilizing, and then mixing with 6 g sodium stearyl fumarate to obtain the particles containing total flavonoids of Desmodium Styracifolium;

tableting the particles containing total flavonoids of Desmodium Styracifolium with a tableting machine so as to obtain 1000 tablets of the pharmaceutical composition in the form of tablets.

### Embodiment 13

### Preparation of the Tablets of Total Flavonoids of Desmodium Styracifolium

### Formula

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 30 | g |
| Lactose | 50 | g |
| sodium Croscarmellose | 20 | g |
| Sodium stearyl fumarate | 5 | g |
| Total | 1000 | tablets |

### Formula: Identical to Embodiment 12

### Embodiment 14

### Preparation of the Soft Capsules Containing Total Flavonoids of Desmodium Styracifolium

### Formula

| Total flavonoids of Desmodium Styracifolium | 133 | g |
|---|---|---|
| Soybean oil | 40 | g |
| Polyoxyethylene (40) hydrogenated castor oil | 80 | g |
| Polyethylene glycol 4000 | 30 | g |
| Pharmaceutical gelatin | 300 | |
| Glycerol | 120 | g |
| Water | 300 | g |
| Total | 1000 | soft capsules |

Preparation was performed by the following steps: preparing a gel solution by immersing gelatin and glycerol in water to swell; mixing 40 g soybean oil, 80 g polyoxyethylene (40) hydrogenated castor oil and 30 g polyethylene glycol 400 in respective formula dosage under stirring or ultrasonic treatment to be uniform, so as to obtain a mixture; dissolving 133 g total flavonoids extract of Desmodium Styracifolium in its formula dosage in the mixture under stirring or ultrasonic treatment at a temperature of 37° C., thereby obtaining content fluids after degassing under vacuum; and filling and compressing content fluids in a soft capsule pelleting machine, thereby obtaining soft capsules; hardening and molding by blow-drying in drum drying equipment, followed by sterilizing and scrubbing with ethanol, natural evaporating the moisture in capsule shell and ethanol for 20 h such that capsule shell is of a dried, smooth and slightly elastic surface, which are subjected to selection for discarding unqualified soft capsules with unqualified appearance and seam and packing qualified soft capsule in a bottle or blister plate, thereby obtaining 1000 capsules containing the pharmaceutical composition in the form of the soft capsules.

### Embodiment 15

### Preparation of Soft Capsules of Total Flavonoids of Desmodium Styracifolium

### Formula

| Total flavonoids of Desmodium Styracifolium | 133 | g |
|---|---|---|
| Soybean oil | 30 | g |
| Polyoxyethylene (40) hydrogenated castor oil | 100 | g |
| Polyethylene glycol 4000 | 20 | g |
| Pharmaceutical gelatin | 300 | |
| Glycerol | 120 | g |
| Water | 300 | g |
| Total | 1000 | soft capsules |

### Preparation: Identical to Embodiment 14

### Embodiment 16

### Preparation of Soft Capsules of Total Flavonoids of Desmodium Styracifolium

### Formula

| Total flavonoids of Desmodium Styracifolium | 66.5 | g |
|---|---|---|
| Soybean oil | 30 | g |
| Polyoxyethylene (40) hydrogenated castor oil | 100 | g |
| Polyethylene glycol 4000 | 20 | g |
| Pharmaceutical gelatin | 300 | |
| Glycerol | 120 | g |
| Water | 300 | g |
| Total | 1000 | soft capsules |

### Preparation: Identical to Embodiment 14

### Embodiment 17

### General Pharmacological Experiments of Total Flavonoids of Desmodium Styracifolium

Experiment object: to observe the pharmacological effect of total flavonoids of Desmodium Styracifolium on general behaviour, state, central nervous system and digestive system of an animal.

Experiment animals and administration: Kunming mice, female, having a weight ranging from 18 g to 22 g, provided by Animal Center of Academy of Military Medical Science with a permit number of experiment animal quality: SOCK (Military) 2002-001, were raised in a mice experiment room of the center with an experiment proved facility number: SYXK (Military) 2002-001.

Experiment grouping: all mice were randomly divided into four groups, i.e., a reference group (administrated with 0.5% sodium carboxymethyl cellulose via gavage), a low-dosage group of total flavonoids of Desmodium Styracifolium (75 mg/kg), a middle-dosage group of total flavonoids of Desmodium Styracifolium (150 mg/kg) and a high-dosage group of total flavonoids of Desmodium Styracifolium (300 mg/kg). Each group contained 10 to 20 mice.

Single gavage was chosen to be the administration route with an administration volume of 0.6 ml/mouse.

Indicators and Results:

1.1 Effects of Total Flavonoids of Desmodium Styracifolium on General Behaviour of Mouse

The general behaviour of mice was observed according to Bastian classification. Each group contained 10 mice, and the observation started after 15 min from gavage for continuous 60 min, which was performed once again after 24 hours. The observation was made to mental, gait, eye, tail, skin, hair and faeces.

After the observation to the general behaviour of the mice, the total flavonoids of Desmodium Styracifolium in the low-, middle- or high-dosage group (75 mg/kg, 150 mg/kg and 300 mg/kg) had little effects on the animal behaviour, action, activity, emotion and gait, with non-significant difference as compared with the reference group.

1.2 Effects of Total Flavonoids of Desmodium Styracifolium on Spontaneous Activity

The results, recorded by a photoelectric method, showed that the spontaneous activities of the mice administrated with different dosages of total flavonoids of Desmodium Styracifolium via gavage had non-significant difference as compared with the reference group, and specific data was shown in Table 9.

TABLE 9
Effect on spontaneous activity of the mice adminitrated with
total flavonoids of Desmodium Styracifolium via gavage

| | | before | | | |
|---|---|---|---|---|---|
| animal | dosage | administration | after administration (times/3 min) | | |
| number | (mg/kg) | (times/3 min) | 30 min | 60 min | 120 min |
| 20 | 0 | 43.5 ± 7.2 | 41.8 ± 3.3 | 40.3 ± 3.1 | 37.8 ± 2.2 |
| 20 | 75 | 43.8 ± 5.0 | 41.3 ± 3.1 | 39.8 ± 3.3 | 38.5 ± 1.3 |
| 20 | 150 | 44.8 ± 5.0 | 39.8 ± 2.2 | 39.3 ± 1.7 | 39.5 ± 1.3 |
| 20 | 300 | 43.5 ± 4.2 | 40.5 ± 1.9 | 39.8 ± 2.2 | 39.5 ± 1.9 |

1.3 Effects of Total Flavonoids of Desmodium Styracifolium on Activating Central Nervous System of Mice

After administrated with total flavonoids of Desmodium Styracifolium via gavage, seizure lasting duration of mouse was observed by subjecting ear tips, applied with an appropriate amount of saline and clamped by a fish-mouth clamp at both sides, to electricity stimulation at a voltage of 110 V for 0.3 second.

As can be seen from obtaining result that the seizure lasting duration caused by the electricity stimulation was not significantly prolonged or shortened by total flavonoids of Desmodium Styracifolium in the low-, middle- or high-dosage group (75 mg/kg, 150 mg/kg and 300 mg/kg) as compared with the reference group; while the seizure occurrence was not changed significantly either (see the specific data shown in Table 10), thus indicating that the total flavonoids had no obvious activating effect on central nervous system via gavage administration.

TABLE 10
Effects on activating central nervous system of the mice administrated
with total flavonoids of Desmodium Styracifolium via gavage

| animal | | dosage | |
|---|---|---|---|
| number | weight (g) | (mg/kg) | seizure lasting duration (sec) |
| 10 | 21.1 ± 0.6 | 0 | 32.8 ± 5.3 |
| 10 | 20.9 ± 0.8 | 75 | 37.1 ± 8.1 |
| 10 | 20.3 ± 0.7 | 150 | 37.7 ± 6.0 |
| 10 | 21.0 ± 0.9 | 300 | 35.5 ± 8.7 |

1.4 Effects of Total Flavonoids of Desmodium Styracifolium on Digestive System of Mice

All mice were randomly grouped such that each group contained 10 mice, all of which were fasten for 12 hours before starting experiment. After 1 hour from administration with total flavonoids of Desmodium Styracifolium, the experiment mice were administrated with a suspending solution made from 5% carbon powder and 10% Arabic gum, with administration volume of 0.2 ml per mouse. All experiment mice were sacrificed after 20 minutes from the administration with the suspending solution for gastrointestinal tract harvest. The gastrointestinal tract was straighten on a glass plate for measuring a distance from pylorus to where the carbon powder headed with a ruler, then a percentage of such the distance to the total length of the gastrointestinal tract was calculated. Obtaining results showed that total flavonoids of Desmodium Styracifolium had no obvious effects on gastrointestinal movement. Specific data was shown in Table 11.

TABLE 11
Effects on propelling rate of the mice administrated with
total flavonoids of Desmodium Styracifolium via gavage

| | total length of | distance from pylorus | |
|---|---|---|---|
| dosage | gastrointestinal | to where the carbon | propelling |
| (mg/kg) | tract (cm) | powder headed (cm) | rate (%) |
| 0 | 52.2 ± 1.9 | 30.2 ± 2.4 | 57.8 ± 4.2 |
| 75 | 52.5 ± 1.7 | 31.6 ± 2.4 | 58.1 ± 3.5 |
| 150 | 52.3 ± 2.3 | 28.8 ± 3.5 | 56.8 ± 5.0 |
| 300 | 52.0 ± 1.9 | 29.8 ± 2.9 | 58.0 ± 3.4 |

### Example 18

### Pharmacodynamic Experiments of Total Flavonoids of Desmodium Styracifolium in Animals

1.1 Experiment of Therapeutic Effects of Total Flavonoids of Desmodium Styracifolium on Ethylene Glycol Calcium Oxalate Kidney Stones in Rats

As compared with the reference group (administrated with 0.5% sodium carboxymethyl cellulose via gavage), the total flavonoids of Desmodium Styracifolium in four dosage groups (50 mg/kg/day, 100 mg/kg/day, 200 mg/kg/day, 400 mg/kg/day) inhibited amount of calcium oxalate crystalline polymer in kidney with a significant dose-effect relationship (P<0.05-0.01); reduced the formation rate of kidney stones (P<0.05-0.01); decreased creatinine content (P<0.05-0.01) and uric acid content (P<0.05-0.01) in serum, and improved kidney function of rats.

1.2 Experiment of Preventing Effects of Total Flavonoids of Desmodium Styracifolium on Ethylene Glycol-Induced Toxic Calcium Oxalate Kidney Stones in Rats

As compared with the reference group (administrated with 0.5% sodium carboxymethyl cellulose via gavage), the total flavonoids of Desmodium Styracifolium in three dosage groups (50 mg/kg/day, 100 mg/kg/day, 200 mg/kg/day) alleviated pyclectasis, reduced the formation rate of kidney stones, decreased the amount of the calcium oxalate crystalline polymer (P<0.01-0.001) and decreased the creatinine content and the uric acid content in serum (P<0.05-0.01).

1.3 Experiment of Dissolving Effect of Total Flavonoids of Desmodium Styracifolium on Implanted Human Bladder Stones in Rats

As compared with the reference group (administrated with 0.5% sodium carboxymethyl cellulose via gavage), total flavonoids of Desmodium Styracifolium in three dosage groups (100 mg/kg/day, 200 mg/kg/day, 400 mg/kg/day) had effects of dissolving stones and reducing the formation of new stones. The total flavonoids of Desmodium Styracifolium in 100 mg/kg/day group lightened the stone weight (P<0.05). The total flavonoids of Desmodium Styracifolium in 200 gm/kg/day group lightened the stone weight (P<0.05) and dissolved 20% stones. The total flavonoids of Desmodium Styracifolium in 400 gm/kg/day group lightened the stone weigh (P<0.01) and dissolved 30% stones.

1.4 Experiment of Diuretic Effects of Total Flavonoids of Desmodium Styracifolium on Rats Suffering Ethylene Glycol-Induced Kidney Stones and Normal Rats

As compared with a reference group (administrated with 0.5% sodium carboxymethyl cellulose via gavage), rats in three dosage groups (50 mg/kg/day, 100 mg/kg/day, 200 mg/kg/day) had a total urine output ranging from 76.4 to 89.5 ml, which was more than that of rats in the normal group (48.1 ml) by 29-36 ml after 6 hours from single administration. After 4 weeks treatment with administration to those rats suffering stones, the urine output within 12 hours was increased significantly, more than that in the model group by 12-36%.

1.5 Experiment of Inhibition Effects of Total Flavonoids of Desmodium Styracifolium on Swelling Degree and Rate Swelling in Rat Toe Injected with Fresh Albumen

As compared with the reference group (administrated with 0.5% sodium carboxymethyl cellulose via gavage), total flavonoids of Desmodium Styracifolium in three dosage groups (100 mg/kg/day, 200 mg/kg/day, 400 mg/kg/day) alleviated the swelling degree and the rate swelling in rat toe injected with fresh albumen, indicating that total flavonoids of Desmodium Styracifolium has a certain anti-inflammatory effect and has an obvious inhibiting effect on proliferation of granulation tissue.

### Example 19

### Acute Toxicity Test of Total Flavonoids of Desmodium Styracifolium in Animals

1.1 Acute Toxicity Tests of Total Flavonoids of Desmodium Styracifolium in Mice

All mice were randomly divided into 6 groups, each containing 20 mice with 10 male and 10 female, with 0.85 distances between groups. After administration, decreased activities, unstable gait, weakened breaths appeared in animals. Most mice died within an hour after the administration, and a few of mice died within 1 to 6 hours after the administration. After calculation by Bliss, LD50 for female was 18.162 g/kg with an upper limit of 20.199 g/kg and a lower limit of 16.326 g/kg under a confidence limit of 95%; LD50 for male was 17.084 g/kg with an upper limit of 18.975 g/kg and a lower limit of 15.301 g/kg under a confidence limit of 95%, with no obvious difference as for LD50 between female and male. According to the results described above, total flavonoids of Desmodium Styracifolium could be recognized as a substantially nontoxic medicament.

1.2 Acute Toxicity Tests of Total Flavonoids of Desmodium Styracifolium in Rats

The test was performed according to “fixed dosage by single oral administration”. Rats were administrated with 2000 mg/kg total flavonoids of Desmodium Styracifolium for preliminary tests, resulting in nonobvious acute toxic reaction; accordingly, 2000 mg/kg was taken as the fixed dosage for formal tests.

Rats for the test were randomly divided into a reference group and an administration group, each containing 10 animals with 5 female and 5 male. Rats in the administration group were administrated with 2000 mg/kg total flavonoids of Desmodium Styracifolium by single gavage, with an administration volume of 2.0 ml/100 g body weight. Rats in the reference group were administrated with 0.5% sodium carboxymethyl cellulose by single gavage, with an administration volume of 2.0 ml/100 g body weight.

Rats in the administration group became lazy to move within 3 hours from the administration; excreted faeces in an ash black color after 1 day from the administration; consumed slightly reduced amount of food, had mildly inhibited increasement in body weight, which recovered to those in the reference group. According to the results described above, total flavonoids of Desmodium Styracifolium could be regarded as a tested medicament without severe and acute toxicity.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A method for preparing a pharmaceutical composition, comprising:
providing alcohol extract of Desmodium Styracifolium containing total flavonoids of Desmodium Styracifolium as an active ingredient; and
adding a pharmaceutically acceptable excipient,
wherein the total flavonoids of Desmodium Styracifolium is of a content ranging from 2.5% to 95 wt % based on a total weight of the pharmaceutical composition,
wherein the alcohol extract of Desmodium Styracifolium is obtained by the following steps:
heating and refluxing a raw material of Desmodium Styracifolium with ethanol having a concentration ranging from 50% to 95% and a weight ranging from 8 to 14 times as heavy as the raw material of Desmodium styracifolium, so as to obtain an extracting solution of Desmodium Styracifolium;
concentrating the extracting solution of Desmodium Styracifolium, so as to remove ethanol; and
subjecting the extracting solution of Desmodium Styracifolium after concentrated to adsorption onto a macroporous resin column, so as to obtain the alcohol extract of Desmodium Styracifolium.

14. (canceled)

15. (canceled)

16. (canceled)

17. The method according to claim 13, wherein the extracting solution of Desmodium Styracifolium is obtained by:
heating and refluxing the raw material of Desmodium Styracifolium for extraction, 1 to 3 times with 1 to 3 hours for each time, with ethanol having the concentration ranging from 50% to 95% and the weight ranging from 8 to 14 times as heavy as the raw material of Desmodium styracifolium, to obtain alcohol extracting solutions of Desmodium styracifolium, and
mixing the alcohol extracting solutions.

18. The method according to claim 13, wherein the alcohol extract of Desmodium Styracifolium is obtained by the following steps:
weighing a raw material of Desmodium Styracifolium, heating and refluxing the raw material for extraction, 1 to 3 times with 1 to 3 hours for each time, at a temperature of 50° C. to 60° C. with ethanol having a concentration of 50% to 95% and a weight ranging from 8 to 14 times as heavy as the raw material, so as to obtain alcohol extracting solutions of Desmodium Styracifolium followed by mixing;
concentrating the alcohol extracting solution to be of a volume 2 to 8 times the weight of the raw material followed by still standing and filtering to obtain a filtrate;
subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and
concentrating the eluted solution into a concentrated solution with a relative density ranging from 1.10 to 1.30 followed by drying and then smashing, so as to obtain the alcohol extract of Desmodium Styracifolium.

19. (canceled)

20. The method according to claim 13, wherein the alcohol extract of Desmodium Styracifolium is obtained by the following steps:
weighing a raw material of Desmodium Styracifolium, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing;
concentrating the alcohol extracting solution to be of a volume 5 times the weight of the raw material followed by still standing and filtering to obtain a filtrate;
subjecting the filtrate to adsorption onto an AB-8 macroporous resin column at a flow rate of 3 column bed volumes per hour, eluting and purifying with water having a volume 10 times the weight of filled resin, and eluting with ethanol having a concentration of 60% and a volume of 8 column bed volumes at a flow rate of 3 column bed volumes per hour, to obtain an eluted solution; and
concentrating the eluted solution to recycle ethanol and to obtain a concentrated solution with a relative density of 1.22 followed by drying under reduced pressure at a temperature of 75° C. and then smashing to obtain the alcohol extract of Desmodium Styracifolium.

21. (canceled)

22. The method according to claim 13, wherein the pharmaceutically acceptable excipient is at least one selected from corn starch, dextrin, lactose, pregelatinized starch, saccharose, microcrystalline cellulose, mannitol, sorbitol, xylitol, calcium hydrophosphate, calcium carbonate, starch paste, hydroxypropyl methyl cellulose, povidone K₃₀, povidone K₂₅, polyethylene glycol 2000, polyethylene glycol 4000, polyethylene glycol 6000, citric acid, succinic acid, dextran, galactose, saccharose, glucose, modified starch, microcrystalline cellulose, poloxamer 188, D-mannitol, methylcellulose, sodium carboxymethyl starch, low-substituted hydroxypropyl cellulose, cross-linked povidone, sodium carboxymethyl starch, croscarmellose sodium, calcium carboxymethyl cellulose, coconut oil amine polyglycol ether, glycerol polyoxyethylene ether, Tween 20, Tween 40, Tween 60, Tween 80, Myrj 40, Brij 30, methoxy polyethylene glycol, sodium dodecyl sulfate, magnesium stearate, talc, aerosil, magnesium dodecyl sulfate, sodium benzoate, and sodium stearyl fumarate.

23. The method according to claim 13, further comprising: formulating the pharmaceutical composition into tablets, effervescent capsules, hard capsules, soft capsules, granules, electuary, pills, or powders.

24. The method according to claim 23, wherein the pharmaceutical composition is formulated into the granules by the following steps:
granulating by spraying, with a gunjet, an adhesion agent towards the alcohol extract of Desmodium Styracifolium and a filler mixed in advance in a fluidized bed, thereby obtaining particles;
drying and discharging the particles to obtain mixed power; and
packaging the mixed power so as to obtain the granules.

25. The method according to claim 23, wherein the pharmaceutical composition is formulated into the granules by the following steps:
preheating the alcohol extract of Desmodium Styracifolium and the pharmaceutically acceptable excipient, sieved at 60 to 100 meshes in advance respectively, to a temperature of 35° C. to 55° C. within 5 min to 60 min in a fluidized bed;
granulating by spraying, with a gunjet under an atomizing pressure ranging from 0.07 MPa to 0.1 MPa and a spraying speed ranging from 15 rpm/min to 25 rpm/min, an adhesion agent into the fluidized bed adjusted with an air inlet temperature of 50° C. to 65° C. to enable materials therein to be of a material temperature between 40° C. to 55° C., by which the adhesion agent is completely sprayed within 5 min to 60 min;
drying resulting particles in the fluidized bed adjusted with an air inlet temperature of 60° C. to 70° C. to enable materials therein to be of a material temperature of 40° C. to 55° C. for 5 min to 60 min;
cooling and discharging the particles to obtain mixed power; and
packaging the mixed power so as to obtain the granules.

26. The method according to claim 23, wherein the pharmaceutical composition is formulated into the granules by the following steps:
dissolving 1 g adhesion agent such as povidone K₃₀ into 120 g water under stirring to be uniform, thereby obtaining an adhesion agent solution for use;
preheating 133 g total flavonoids extract of Desmodium Styracifolium, 110 g microcrystalline cellulose and 60 g lactose, mixed in advance, to a temperature of 45° C. within 20 min in a fluidized bed;
granulating by spraying, with a gunjet under an atomizing pressure of 0.09 MPa and a spraying speed of 20 rpm/min, the adhesion agent solution into the fluidized bed adjusted with an air inlet temperature of 55° C. to enable materials therein to be of a material temperature of 45° C., by which the adhesion agent solution is completely sprayed within 15 min;
drying resulting particles in the fluidized bed adjusted with the air inlet temperature of 65° C. to enable materials therein to be of the material temperature of 45° C. for 10 min;
cooling and discharging the particles to obtain mixed power, and
packaging the mixed power so as to obtain 1000 bags of the pharmaceutical composition in the form of the granules.

27. The method according to claim 23, wherein the pharmaceutical composition is formulated into the hard capsules by the following steps:
mixing the alcohol extract of Desmodium Styracifolium and a filler in a fluidized bed;
granulating by spraying, with a gunjet, an adhesion agent into the fluidized bed;
drying and discharging resulting particles to obtain mixed power; and
capsulizing the mixed power with an encapsulating machine, so as to obtain the pharmaceutical composition in the form of the capsules.

28. The method according to claim 23, wherein the pharmaceutical composition is formulated into the hard capsules by the following steps:
preheating the alcohol extract of Desmodium Styracifolium and the pharmaceutically acceptable excipient, sieved at 60 to 100 meshes in advance respectively, to a temperature of 35° C. to 55° C. within 5 min to 60 min in a fluidized bed;
granulating by spraying, with a gunjet under an atomizing pressure ranging from 0.07 MPa to 0.1 MPa and a spraying speed ranging from 15 rpm/min to 25 rpm/min, an adhesion agent into the fluidized bed adjusted with an air inlet temperature of 50° C. to 65° C. to enable materials therein to be of a material temperature between 40° C. to 55° C., by which the adhesion agent is completely sprayed within 5 min to 60 min;
drying resulting particles in the fluidized bed adjusted with an air inlet temperature of 60° C. to 70° C. to enable materials therein to be of a material temperature of 40° C. to 55° C. for 5 min to 60 min;
cooling and discharging the particles to obtain mixed power, and
capsulizing the mixed power, so as to obtain the hard capsules.

29. The method according to claim 23, wherein the pharmaceutical composition is formulated into the hard capsules by the following steps:
dissolving 1 g adhesion agent such as povidone K₃₀ into 120 g water under stirring to be uniform, thereby obtaining an adhesion agent solution for use;
preheating 133 g total flavonoids extract of Desmodium Styracifolium, 30 g microcrystalline cellulose and 37 g lactose, mixed in advance, to a temperature of 45° C. within 20 min in a fluidized bed;
granulating by spraying, with a gunjet under an atomizing pressure of 0.09 MPa and a spraying speed of 20 rpm/min, the adhesion agent solution into the fluidized bed adjusted with an air inlet temperature of 55° C. to enable materials therein to be of a material temperature of 45° C., by which the adhesion agent solution is completely sprayed within 15 min;
drying resulting particles in the fluidized bed adjusted with the air inlet temperature of 65° C. to enable materials therein to be of the material temperature of 45° C. for 10 min;
cooling and discharging the particles to obtain mixed power; and
capsulizing the mixed power with an encapsulating machine, so as to obtain 1000 hard capsules of the pharmaceutical composition.

30. The method according to claim 23, wherein the pharmaceutical composition is formulated into sugar-coated tablets or film-coated tablets by the following steps:
mixing the alcohol extract of Desmodium Styracifolium, a solid dispersion carrier and a surfactant with 50% ethanol followed by heating and stirring for dissolution, removing the solvent via evaporation under reduced pressure, vacuum drying, smashing and sieving, so as to obtain solid dispersion containing total flavonoids of Desmodium Styracifolium;
mixing a filler and a disintegrant with the solid dispersion containing total flavonoids of Desmodium Styracifolium to be uniform followed by granulating, drying and size stabilizing, and then mixing with a lubricant to be uniform, so as to obtain particles containing total flavonoids of Desmodium Styracifolium;
tableting the particles containing total flavonoids of Desmodium Styracifolium with a tableting machine so as to obtain tablets; and
coating the tablets with sugar or films, so as to obtain the sugar-coated tablets or film-coated tablets.

31. The method according to claim 23, wherein the pharmaceutical composition is formulated into sugar-coated tablets or film-coated tablets by the following steps:
mixing the alcohol extract of Desmodium Styracifolium, a solid dispersion carrier and a surfactant, sieved at 40 to 200 meshes in advance, with 50% ethanol under stirring and heating to a temperature of 50° C. to 75° C. for dissolution, removing the solvent via evaporation under reduced pressure at a temperature of 30° C. to 75° C., vacuum drying at a temperature of 30° C. to 60° C., and then smashing and sieving at 40 to 200 meshes so as to obtain the solid dispersion containing total flavonoids of Desmodium Styracifolium;
mixing a filler and a disintegrant, sieved at 40 to 100 meshes in advance respectively, with the solid dispersion containing total flavonoids of Desmodium Styracifolium to be uniform, preparing a soft material, granulating at 10 to 30 meshes, drying at a temperature of 30° C. to 75° C., size stabilizing, and then mixing with a lubricant to be uniform, thereby obtaining the particles containing total flavonoids of Desmodium Styracifolium;
tableting the particles containing total flavonoids of Desmodium Styracifolium with a tableting machine so as to obtain the tablets; and
coating the tablets with sugar or films, so as to obtain the sugar-coated tablets or film-coated tablets.

32. The method according to claim 23, wherein the pharmaceutical composition is formulated into sugar-coated tablets or film-coated tablets by the following steps:
mixing 66.5 g the alcohol extract of Desmodium Styracifolium, 266 g povidone K₃₀, 133 g poloxamer 188 and 39.9 g sodium dodecyl sulfate, sieved at 80 meshes in advance, with 50% ethanol, stirring and heating to a temperature of 65° C. for dissolution, removing the solvent via evaporation under reduced pressure at a temperature of 50° C., vacuum drying at a temperature of 40° C., and then smashing, so as to obtain the solid dispersion containing total flavonoids of Desmodium Styracifolium;
mixing 10 g lactose and 15 g sodium croscarmellose, sieved at 80 meshes in advance respectively, with the solid dispersion containing total flavonoids of Desmodium Styracifolium to be uniform, preparing a soft material, granulating at 20 meshes, drying at a temperature of 55° C., size stabilizing, and then mixing with 6 g sodium stearyl fumarate to obtain the particles containing total flavonoids of Desmodium Styracifolium;
tableting the particles containing total flavonoids of Desmodium Styracifolium with a tableting machine so as to obtain 1000 tablets; and
coating the tablets with sugar or films, so as to obtain the sugar-coated tablets or film-coated tablets.

33. The method according to claim 23, wherein the pharmaceutical composition is formulated into the soft capsules by the following steps:
mixing an oil phase, a surfactant and a co-surfactant to be uniform under stirring or ultrasonic treatment to obtain a mixture;
dissolving the alcohol extract of Desmodium Styracifolium into the mixture under stirring or ultrasonic treatment, and
capsulizing into a soft capsule to obtain the soft capsules.

34. The method according to claim 23, wherein the pharmaceutical composition is formulated into the soft capsules by the following steps:
mixing 40 g soybean oil, 80 g polyoxyethylene (40) hydrogenated castor oil and 30 g polyethylene glycol 400 in respective formula dosage under stirring or ultrasonic treatment to be uniform, so as to obtain a mixture;
dissolving 133 g total flavonoids extract of Desmodium Styracifolium in its formula dosage in the mixture under stirring or ultrasonic treatment at a temperature of 37° C., thereby obtaining content fluids after degassing under vacuum; and
filling and compressing content fluids in a soft capsule pelleting machine, thereby obtaining 1000 soft capsules.

35. A pharmaceutical composition, prepared by a method according to claim 13.

36. A method for treating urinary stone, comprising administrating the pharmaceutical composition prepared by a method according to claim 13 to in a subject in need thereof.
